(19) United States
(12) Patent Application Publication
(10) Pub. No.: US 2004/0268421 A1
(43) Pub. Date: Dec. 30, 2004

(54) Animal model for medical device testing and training using xenografted organ structures such as blood vessels

(75) Inventors: Gary J. Tearney, Cambridge, MA (US); Jay D. Caplan, Belmont, MA (US)

Correspondence Address: HOUSTON ELISEEVA, 4 MILITIA DRIVE, SUITE 4, LEXINGTON, MA, 02421, US

(73) Assignee: InfraReDx, Inc., Cambridge, MA

(21) Appl. No.: 10/610,318

(22) Filed: Jun. 30, 2003

(51) Int. Cl. A01K 67/00

(52) U.S. Cl. 800/3; 800/9

(57) ABSTRACT

A method for assessing medical instrumentation having a probe, such as a catheter, is based on the process of inserting the catheter into a non-human animal having a human tissue graft, such as a coronary artery graft exhibiting an atherosclerotic lesion. The catheter is then moved to the human tissue graft. Then, an analysis or treatment of the human tissue graft is performed using the catheter. The performance of the medical instrumentation can be thus assessed relative human tissue. The testing is performed on a live animal, thus creating a physiologic and biomechanical environment similar to that found in a human, without the necessity of human testing.

### BACKGROUND OF THE INVENTION

Animal models are typically used by scientists to gain a better understanding of the mechanisms underlying disease. An animal model for biomedical research is typically one in which a spontaneous or induced pathological process can be investigated, and in which the medical condition in one or more respects resembles the same condition in humans.

Developing an animal model typically requires tradeoffs between the model's feasibility and the ability of the model to shed insight into the condition in humans. Sometimes xenografting techniques are used to improve the correlation between the animal model and human trials, since the subsequent tests can be performed on human tissue exhibiting the condition of interest, rather than animal tissue that resembles human tissue in only some respects. Moreover, it can be relatively easy to perform the surgical procedure associated with the xenograft, making this approach more feasible that culturing or inducing the condition in the animals, which may require a long time period.

For example, immune-incompetent nude mice are used for in vivo study of human tumors to evaluate therapeutic agents or methods of imaging the tumors. Human tumors are xenoplanted subcutaneously into the mice. The agents are then administered to the mice, and the affect on the tumor or the ability to image the tumor is gauged.

Others have proposed to xenograft airway cells from a human respiratory system into non-human animals for testing. The diseased airway and the animal that carries it are useful as models for cystic fibrosis and for assaying the effects of various therapies and agents.

Animal models are also required in the testing of diagnostic and therapeutic tools or devices. For example, new catheter-based systems are applicable to a number of medical applications. Catheter-based optical systems include optical coherence tomography, which is used to provide spatial resolution, enabling the imaging of internal structures. Spectroscopy is used to characterize the composition of structures, enabling the diagnosis of medical conditions, by differentiating between cancerous, dysplastic, and normal tissue structures, for example. Exemplary therapeutic systems include ablation systems that are used to remove or destroy structures within the body to address various diseases, such as tachycardias, tumors, and coronary artery disease.

These catheter-based systems enable minimally invasive diagnosis and/or treatment. However, before they are used in human trials, it is often desirable to first test their efficacy and safety on animal subjects.

For example, in one specific spectroscopic application, generated light in near infrared or 850 nanometers (nm) to 1-2 micrometers (μm), for example, is used to illuminate tissue in a target area in vivo using the catheter. Diffusely reflected light resulting from the illumination is then collected and a spectral response of the tissue identified. The response is used to assess the composition or state of the tissue.

One use of this near infrared spectroscopic system is the diagnosis of atherosclerosis, and the identification of atherosclerotic lesions or plaques. This is an arterial disorder involving the intimae of medium- or large-sized arteries, including the aortic, carotid, coronary, and cerebral arteries. Efforts are being made to spectroscopically analyze blood vessel walls and characterize the composition of any atherosclerotic lesions.

A number of animal models have been considered for these catheter-based diagnostic and therapeutic systems, especially for arterial disorders. New Zealand rabbits grow sclerotic lesions. Unfortunately, the small size of this rodent presents difficulties when testing catheter systems scaled for human subjects. Moreover, the plaques can require time to develop in these rabbits, impacting feasibility, and the plaques may not resemble human plaques to the degree required to test spectroscopic diagnostic systems, which are responsive to the plaque composition.

Porcine models have problems similar to those of the New Zealand rabbits. Time is required to develop the lesions in the pigs, and the resemblance to humans is unclear. On the other hand, catheters sized for human subjects will typically fit into pig blood vessels.

Techniques have been proposed to accelerate the process of lesion formation in porcine models to improve feasibility. One strategy relies on induced injuries, using balloon catheters, which are inflated at the site where the lesion is desired. Injections into the arterial walls have also been proposed. The concern, however, is that these models will not yield lesions that have adequate similarities to the lesions in humans, as required for the spectroscopic applications, for example.

A good animal model is important in the testing of these catheter-based optical systems because there are a number of challenges associated with the biomechanics that render testing on cadavers incomplete. To be medically relevant, the system must be able to function in the presence of flowing blood and cyclic movement between the catheter head and the artery walls due to the motion of the beating heart. This environment cannot be accurately duplicated in a cadaver or in some ex vivo test environment that includes cadaver artery tissue.

### SUMMARY OF THE INVENTION

In applications such as diagnostic and therapeutic instrumentation testing, including implant such as stents, and also drug testing, it is many times necessary to closely simulate the human disease state while also duplicating the biomechanics of organ systems that affect the operation of the instrumentation or the affect of the drugs. In such cases, a pure animal model, while having organ function, may not provide an accurate enough simulation of the human disease state and/or creating the disease state in the animal may not be feasible, requiring too much time. On the other hand, testing on the cadaver may be inadequate because the lack of organ function precludes the construction of an environment simulating the physiology (e.g., temperature, extracellular fluid composition) and biomechanics (e.g., motion, flow, strain) of the human patient.

In the case of testing intra-vessel instrumentation, such as catheters for insertion into the coronary arteries to detect atherosclerosis, or the testing of stents, the motion associated with the beating heart, in combination with the catheter head's or stent's interaction with the flowing blood affects the relationship with the walls of the artery that are being assessed or treated. Similar issues arise when testing endoscopes in the colon due to motion in the digestive track, for example. As a result, in these applications, it is difficult to obtain good performance data from cadavers, which have no organ function, or pure animal models in which any vascular lesions or intestinal abnormalities may be chemically or visually different from similar features of interest in humans. Thus, a need exists for a good animal model and a method for using or testing such instrumentation or drugs.

The present invention is directed to an animal model and methods for medical testing. It uses non-human animals into which human tissue has been xenoplanted. In one example, human blood vessels are used. This enables animal-based testing but testing relative to human tissue to thereby better simulate the human subject.

This animal model is useful in the testing of the safety of the instrumentation. For example, interventional cardiology devices may cause morbidity due to unintended effects. Specifically, a new diagnostic catheter may unintentionally injure the blood vessel walls due to excess forces. To test this, the catheter is preferably tested on an animal by performing the procedure and then sacrificing the animal. A subsequent histological examination of the vessel is performed to assess the performance, such as safety of the instrumentation. In another example, atherectomy catheters for the removal of plaque tissue are tested in the animal model. However, it may not be possible for the operator to prevent perforations of the artery. To test this, the procedure is performed on the animal model with human blood vessel grafts, and any perforations are detected by one or more ways. For example, angiography can be performed, and leakage detected. Alternately, sacrifice and histology can be performed. The inventive animal model provides a more realistic simulation of the human atherectomy environment.

As another example, to test whether a new stent stimulates the unwanted growth of intimal tissue, the procedure is performed in the inventive animal model and the growth of intimal tissue is followed over time by intravascular ultrasound (IVUS) or optical coherence tomography (OCT), or angiography. Subsequent sacrifice and histology are used to determine the intimal tissue growth.

The animal model also has relevance to training. It is known that physicians and hospitals that perform higher procedure volumes for coronary stenting have better outcomes and reduced mortality/morbidity. In order to improve outcomes, physicians and catheter laboratory personnel can use the animal model as a vehicle for practicing standard stent placement techniques on animals. Optionally, histological examination can be performed afterward to look for possible problems such as stents not being placed properly in the lesion. This training can also be used for new operators.

The animal model is also useful in the testing of agents. In one example, drugs are delivered locally by interventional devices such as drug delivery catheters or stents. There may be interaction between agent and the delivery device or method, meaning that the same agent may be more or less effective depending on how it is delivered. Second, drugs may be designed to be activated by interventional devices such as light emitting catheters for photodynamic therapy agents, or heat emitting catheters for liposome-encapsulated agents. Again, there may be interaction between agent and the activating device or method, meaning that the same agent may be more or less effective depending on how it is activated. Third, disease progression is affected by the hemodynamic and environmental conditions in the blood vessel. Getting the pressures, flow, and other variables to track those in the human patient is often important. Consequently, evaluation of statins for the reduction of high risk plaques is best done in a model which simulates the environmental, mechanical, and hemodynamic conditions to which the artery would normally be subject. In each of these examples, the inventive animal model provides a mechanism for testing the agents relative to human tissue without concomitant risk to human subjects.

Therefore, in general, according to one aspect, the invention features a method for using medical instrumentation that has a probe, such as a catheter. The method comprises inserting the probe into a non-human animal having a human tissue graft. The probe is then moved to the human tissue graft. Then, an analysis or treatment of the human tissue graft is performed using the probe.

In some cases, the method is used to assess a safety and/or performance of the medical instrumentation. In other cases, the method is used as a vehicle for training operators of the medical instrumentation.

In the typical embodiment, a performance of the medical instrumentation is assessed. One advantage of the invention is that the operation of the medical instrumentation can be assessed relative to human tissue, but in the context of an animal model. In the present implementation, the human tissue that is grafted into the non-human animal is a blood vessel segment. Typically, it is a blood vessel segment that exhibits an atherosclerotic lesion. The blood vessel segment can be attached as a bypass graft to the heart of the non-human animal. This allows the treatment and/or analysis of the tissue graft in an environment that closely resembles that which would be present in a living, human patient.

Preferably, the probe is moved through the non-human animal, as it would be in a human patient. For example, in the case of assessing the state of coronary arteries, the probe is moved through the blood vessels of the non-human animal to the human tissue graft. In the present embodiment, the step of analyzing and/or treating the human tissue graft using the probe comprises detecting a spectral response from the human tissue. It also can comprise a treatment of the tissue graft, such as by exposing the tissue to optical energy.

In general, according to another aspect, the invention features an animal model for the testing of medical instrumentation having a probe. This animal model comprises live non-human animals into which human blood vessels have been grafted.

In the present embodiment, these human blood vessels are coronary arteries that exhibit atherosclerotic lesions. The blood vessel segments are grafted onto the heart of the non-human animal as a bypass graft in the present example. Preferably, in the preferred embodiment, human tissue surrounding the blood vessels is also included to thereby duplicate the optical environment in humans. As a result, the surrounding layers of epicardial fat can be maintained.

In general, according to another aspect, the invention features an animal model comprising live non-human animals into which human vascular tissue have been grafted. This allows for testing relative to vascular tissue to assess diseases associated with the cardiovascular system for example.

In general, according to another aspect, the invention features an animal model comprising live non-human animals into which human cadaver tissue has been grafted. The advantage associated with cadaver tissue is that it is easy to obtain.

In general, according to another aspect, the invention features an animal model comprising live non-human animals into which human organ structures have been orthotopically grafted. Orthotopic grafting creates an environment that is very similar to the environment in humans.

In general according to another aspect, the invention features an animal model comprising live non-human animals into which human organ structures have been heterotopically grafted to simulate motion associated with a cardiovascular system. Heterotopic grafting can be used to ease the medical procedures associated with the graft, for example, improving animal survivability.

In general according to another aspect, the invention features a medical testing method comprising resecting a human tubular organ structure and grafting the human tubular organ structure into a non human animal and then performing medical probe testing on the human tubular organ structure in the non human animal. This method enables probe testing relative to human tissue, but in an animal.

In general according to another aspect, the invention features a medical testing method that comprises resecting human vascular tissue and grafting the human vascular tissue into a non human animal, and performing medical testing on the human vascular tissue in the non human animal. This method enables testing relative to human tissue vascular, but in an animal.

In general according to another aspect, the invention features a medical testing method comprising resecting human cadaver tissue. The human cadaver tissue is then grafted into a non human animal and medical testing performed on the human cadaver tissue in the non human animal. The advantage associated with cadaver tissue is that it is easy to obtain.

In general according to another aspect, the invention features a medical testing method comprising resecting a human organ structure, orthotopically grafting the human organ structure into a non human animal, and performing medical testing on the human organ structure in the non human animal. Orthotopic grafting creates an environment that is very similar to the environment in humans.

In general according to another aspect, the invention features a medical testing method comprising resecting a human organ structure, heterotopically grafting the human organ structure into a non human animal to simulate motion associated with a cardiovascular system, and performing medical testing on the human organ structure in the non human animal. Heterotopic grafting can be used to ease the medical procedures associated with the graft, for example, improving animal survivability.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1A is a flow diagram illustrating the application of the inventive animal model to assess medical instrumentation according to the present invention;

FIG. 1B is a flow diagram illustrating the application of the inventive animal model to assess chemical agents, such as drugs, according to the present invention;

FIG. 2 is a plan view showing the relationship between the catheter head and the artery walls for a spectroscopic analysis system to which the present invention is applicable in one embodiment;

FIG. 3 is a flow diagram illustrating the detailed steps of the resection of the organ structure from the cadaver in a preferred embodiment;

FIG. 4 is a flow diagram illustrating the preparation of the resected artery, according to the preferred embodiment;

FIG. 5 is a flow diagram illustrating the xenoplantation of the human blood vessel artery segment, in the preferred embodiment;

FIG. 6 is a schematic diagram of a pig and its circulatory system with a orthotopic graft of human coronary artery segment and a heterotopically grafted heart from another pig, which has a resected human coronary segment according to embodiments of the inventive animal model; and

FIG. 7 is a schematic diagram of the pig heart with the resected human cadaver coronary segment of the inventive animal model according to the preferred embodiment.

### DETAILED DESCRIPTION OF THE PREFERRED EMBODIMENTS

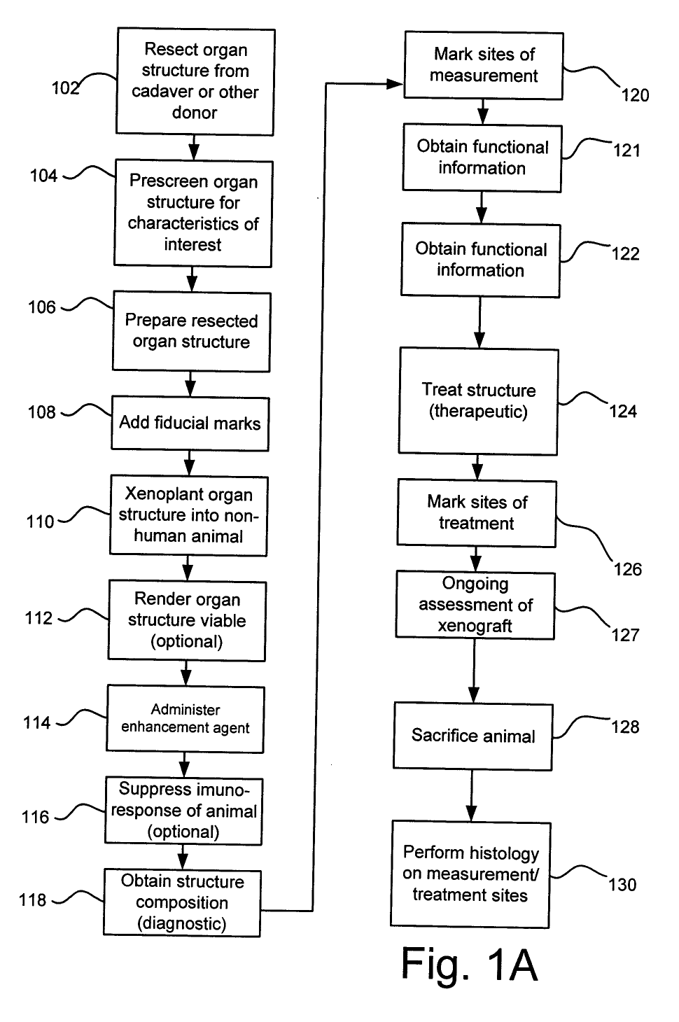

FIG. 1A illustrates a method for assessing probe-based, and specifically catheter-based, medical instrumentation and/or agents using the inventive animal model, which has been defined according to the principles of the present invention.

Specifically, tissues that are functionally or physically associated, such as tissues of an organ structure, are resected from a cadaver or other human tissue donor in step 102. In the typical example, this organ structure includes diseased tissue. Although in other embodiments, normal tissue or organ structures are used.

Another possible donor source is surgical specimens. Surgical specimens are specimens that are resected from patients to obtain a diagnosis of disease. Often, a large portion of each surgical specimen is not used for diagnosis and is therefore available for grafting as described herein.

A major advantage of a surgical specimen is that it is typically recently excised (minutes to hours) and is not as likely to have evidence of tissue degradation that may be present in autopsy tissues.

Surgical coronary specimens are unusual, however. Other arteries with atherosclerosis such as femoral arteries from leg amputations are much more common.

In most examples, the resected organ structures are tubular tissue structures from one of the major body organs. In the present embodiment, the organ structures are conduits or vessels such as arteries, veins, or lymphatic vessels, and specifically coronary artery blood vessels that exhibit atherosclerotic lesions. Specific examples of arterial vessels include the coronary arteries, carotid arteries, aorta, vertebral, basilar, cerebral, femoral, and iliac arteries. Examples of important venous vessels include saphenous, popliteal, femoral, and jugular veins.

In other examples, other tubular organ structures are resected to be analyzed or treated by the medical instrumentation or chemical agents. For example, a section of the esophagus, stomach, small intestine, large intestine, anus, or rectum is resected from the tissue donor. In still other examples, the nasopharynx, pharynx, larynx, bronchial tubes are resected. Other portions of the genital or urinary may be resected, including vagina, urethra, cervix, uterus, fallopian tube, ureters, and renal pelvis. Alternative conduits such as Eustachian tube, biliary duct, pancreatic duct, salivary ducts, are also resected in other applications.

In step 104, pre graft assessments of the tissue or organ structures are preferably made for the relevant characteristics. The structures are pre-screened prior to grafting to assure that the desired tissue types or features are present.

In the specific example of diagnosing atherosclerosis in vascular organ structures, the arteries are pre-screened for sections exhibiting lipid-rich lesions. This can be performed using technologies such as intravascular ultrasound (IVUS) or optical coherence tomography (OCT) to ensure that the grafted artery contains a lipid pool having the desired characteristics.

Alternatively, in another specific example of assessing stent deployment and performance, arteries are prescreened for calcific plaques for subsequent t xenoplantation. Furthermore, if there was a particular landmark or feature, such as a bifurcation of the artery or particular artery diameter, that needed to be tested, this is also identified in the prescreening step.

Next, in step 106, the resected tissue or organ structure is prepared for xenoplantation. In many typical examples, the ends of the tubular organ structure are prepared so that they may be easily connected to the non-human animal. Additionally, extraneous tissue is generally removed to ease the xenoplantation. On the other hand, care must be taken so that tissue removal does not change the response or performance of the instrumentation to be tested.

In step 108, registration relative to histology is preferably achieved by application of fiducial markers to the organ structures. Specifically, the tissue or organ structures are altered prior to grafting, or possibly after grafting while the graft is exposed, to provide fiducial markers that can be used to register an imaging technique with the advancement of the probe through the organ structure, for example and possibly further with the possible subsequent histopathology.

For example, in one implementation, radio-opaque clips are applied to the organ structure that are visible by cineangiography and/or computed tomography (CT). These markers then register the CT with the specimen so that histopathology is conducted at or very near the sites where data are collected from the probe or catheter.

In step 110, the organ structure is xenoplanted into the non-human animal. In the preferred example, the xenoplantation is performed into a dog or a pig to yield a canine or porcine animal model. In other examples, monkeys are used. Generally, it is important that a large animal is selected since in the testing of instrumentation and even some agents, the size of the organ structure impacts how the instrumentation performs.

In step 112, the xenoplanted organ structure is rendered viable. This is typically achieved by connecting supporting vasculature to the animal's circulatory system. Nerves associated with the structure are also connected in some applications.

However, this is an optional step. If the animal model is only being used for diagnostic purposes, then the long term viability of the organ structure is often not required. In contrast, if functional information is desired to test therapeutic instrumentation or agents, then many times it is required to render the organ structure viable in order to test the long-term results of the treatment or impact of the medical instrumentation on the animal model.

In many examples viability is not required and non-viability actually provides some benefits. That is, non-viable human tissue or organ structures are readily available from cadavers. This type of tissue tends to be easier to obtain for medical testing. Thus, by avoiding the need to have viability, the model becomes significantly more feasible to implement.

In some embodiments, enhancement agents are administered in step 114. Typically when the xenograft is rendered viable and the circulation of the animal was connected to the xenograft, it is possible to administer photodynamic (PDT) agents, exogenous chromophores, magnetic resonance imaging (MRI) contrast agents, ultrasound contrast agents, and/or other exogenous signal enhancement agents that will improve the performance of the instrumentation, or enable the testing of the performance of the agent itself, using this animal model. One further example is a detectable lipid-avid agent that is administered to the animal in a diagnostic amount. This agent is allowed to penetrate through the grafted organ structure, such as blood vessel, and bind to the oxidized LDL-cholesterol. The unbound agent is allowed to clear from the animal. This agent is used to facilitate the detection of the lesions by detection of the agent.

Typically, the enhancement agent is administered systemically into the animal, with uptake into the xenograft. Then the “exogenous chromophore enhanced” diagnosis/therapy is assessed, for example.

If using surgical specimens from living patients, it is sometimes possible to deliver an exogenous chromophore prior to surgery, then detect the chromophore (e.g. using fluorescence spectroscopy) or treat the tissue with PDT or chromophore-enhanced ablation. Alternatively, the patient is given a radio-isotope or MRI/ultrasound contrast agent that is then subsequently detected.

In one specific example of photodynamic therapy (PDT) a light sensitive agents such as porphyrins are administered. Porphyrins absorb energy from photons and transfer this energy to surrounding oxygen molecules. Toxic oxygen species such as singlet oxygen and free radicals are thus formed. These chemicals are very reactive and can damage proteins, lipids, nucleic acids and other cellular components to improve the efficacy of the therapy.

In step 116, the immuno-response of the animal is suppressed. This is another optional step, however. Whether the step is performed is dependent upon the time frame over which the animal must be viable in the animal model and whether the animal is already immuno incompetent. In the typical example, the immuno-response of the animal is suppressed with cyclosporine. This will help to inhibit hyper acute and acute rejection of the xenoplanted organ structure. In any event, heparin is typically administered to prevent clotting when the xenoplantation involves the animal's circulatory system due to the corrupted epithelium, which will otherwise give rise to clotting.

In step 118, the test of the agent and/or medical instrumentation is started. Specifically, in the case of probe-based medical instrumentation, the probe, such as a catheter, is inserted into the non-human animal and advanced to the site of the xenoplanted organ structure. In the present implementation, the structural composition of the organ structure is determined using the instrumentation. In other examples, the performance of the enhancement agent is assessed.

In the present embodiment, near infrared spectroscopy instrumentation is tested relative to the xenograft. In other embodiments, fluorescence imaging (e.g. illuminating the tissue with a band of light, and imaging in the fluorescent band) or fluorescence spectroscopy instrumentation is tested.

The model is also applicable to the testing of other systems, however. For example, instrumentation for intravascular imaging, intravascular elastography, palpography, raman spectroscopy, magnetic resonance spectroscopy, and spectral analysis of ultrasonic signals are tested in other applications of the animal model.

The present animal model further has relevance to medical personnel training. For example, interventionalists are trained on the animal model in the deployment of stents or how to conduct intravascular procedures or diagnoses using the xenografted artery or other organ structure in still further applications.

In one specific example, the interposition of a human coronary segment provides a better training setting than the native porcine coronary due to the presence of human diseased tissue.

The animal model is further relevant to the testing of and training on non-invasive imaging technologies. These include ultrasound imaging (US), computed tomography (CT), magnetic resonance imaging (MRI), as the artery could be harvested after testing for histopathologic confirmation of the findings of the imaging modality. Examples of where this is particularly important are MRI and CT, where cardiac motion causes problems because the imaging time is typically long relative to the cardiac cycle. The animal model is useful in the testing of new techniques for overcoming artifacts caused by cardiac motion.

Preferably, the places at which the composition is assessed in step 118 are marked in step 120. This facilitates subsequent histological analysis. For example, in the case of assessing the structural composition of a xenoplanted cadaver coronary artery segment, sutures are placed at the locations where the medical instrumentation is used to assess the structural composition and specifically the existence of atherosclerotic lesions, or not.

In step 121, the xenografted organ structure is monitored. For example, in the case of a xenografted blood vessel, the blood flow through the vessel and the blood pressure in the vessel are monitored either externally or through embedded transducers. This monitoring is sometimes critical to assessing the correspondence between the xenograft and human physiology, i.e., accuracy of the animal model. Simultaneous electrocardiogram (ECG) monitoring is also used to evaluate the diagnostic tool in the graft as a function of cardiac cycle. These techniques improve the ability to relate the model to human physiologic conditions.

In step 122, if the organ structure is rendered viable, then functional information is obtained using the medical instrumentation. Typically, information such as the viability of the xenoplanted organ structure is acquired using the medical instrumentation.

In step 124, if the medical instrumentation is therapeutic in nature, the xenoplanted organ structure is treated. An example in this case would be where the medical instrumentation to be assessed performs optical treatment, such as ablation, of structures in the artery, intestines, or GU tract. In another example, the probe is used to place a stent in the artery.

Again, in step 126, the sites of the treatment are marked. Here again, this is useful for subsequent histological analysis.

In step 127, typically in the situations in which the xenografted organ structure is rendered viable, ongoing assessments of the organ structure are made over the course of the experiment. For example, when studying the long term effects of a therapy or therapeutic agent, such as statins, tissue assessment is performed periodically using angiography or IVUS, as is done in human trials.

In step 128, the animal is typically sacrificed. This allows the final step 130 where the sites of the measurement and/or treatment are excised and subject to further analysis. In the case of measuring the structural composition, the results obtained from the medical instrumentation are compared to the histological, histopathologic analysis of the sites. In the case of therapeutic instrumentation, the success of the treatment performed by the instrumentation is assessed by analysis of the treatment sites.

FIG. 1B shows an embodiment of the medical testing method for the testing of agents such as drugs using the inventive animal model.

Many of the steps are the same as those described relative to FIG. 1A. Some differences typically exist, however.

For example, in drug testing viable grafts are typically desired or in fact required since the time horizons are much longer. Thus, the establishment of viability in step 112 and the administration of immunosuppressants in step 116 are not optional.

Then in step 124-D, the chemical agents or drugs, which are typically therapeutic, are administered. In one example, the statin is administrated in dosages that are appropriate for the animal's size and weight.

Then, in step 127-D then effect of the agents on a xenoplanted tissue or organ structure is assessed in vivo. Any of the previous described modalities for assessment can be used in this step. Most commonly for assessing blood vessels, angiography or IVUS is used. This assessment is typically repeated on a periodic basis and the administration of the agent may be ongoing depending on the specific testing protocol.

The animal sacrifice of step 128 involves certain percentages of the subject population in some implementations to track the effect of the agent on the graft over time via the histology step of 130. Although, in some testing protocols, the entire testing population is sacrificed at the end of the study.

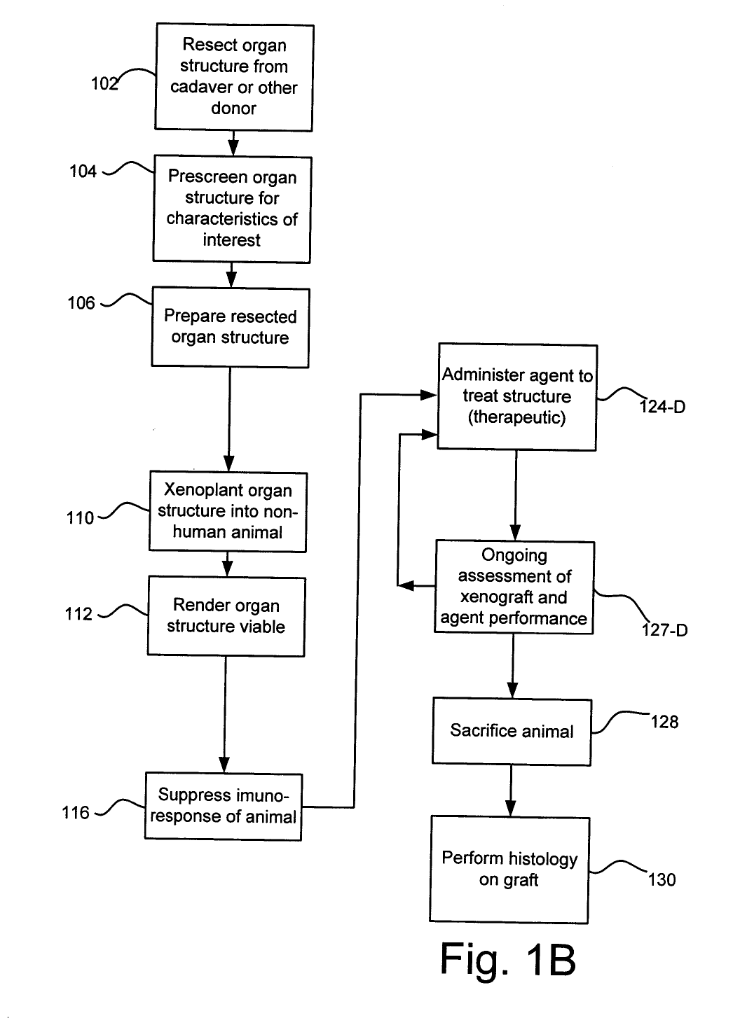

FIG. 2 shows a catheter-based medical optical system to which the present invention is applicable in one example.

The specific, illustrated probe or catheter system 56 is used for spectroscopic analysis of the intimae of blood vessels to find atherosclerotic lesions or plaque.

In the current embodiment, the catheter 56 that includes an optical fiber or optical fiber bundle. The catheter 56 is typically inserted into the non-human animal. This can be accomplished via a peripheral vessel, such as the femoral artery, when analyzing blood vessels. The catheter head 58 is then moved to a desired target area, a xenoplanted coronary artery, for example.

In other applications, the probe is moved through the body 2, other than through blood vessels. For example, the inventive animal model and method is also applicable to the testing of laparoscopes, angioscopes, arthroscopes, colposcopes, sigmoidscopes, colangioscopes to list a few. Generally, the present invention is applicable when an animal model is required for testing or training but the testing needs to be performed or is better when performed relative to human tissue.

When in the xenoplanted artery, radiation, such as an optical signal, 102 from the optical fiber of the catheter 56 is directed by a fold mirror 62, for example, to exit from the catheter head 58 via an output or catheter window 48 and impinge on the target area 22 of the artery wall 24 of the xenoplanted tissue. In the current example, the catheter head 58 then collects reflected and scattered radiation from the target area 22 through the same window 48. The radiation is transmitted to a detector, which performs spectroscopic analysis of the radiation to thereby determine the composition of the target area 22.

An advantage of the present invention is that this testing is performed on live, functioning animal. Thus, the performance of the system and specifically catheter head 58 is tested in an environment including flowing blood 108, which may induce movement 104 in the head 58 and movement due to the function of the animal's organ systems that induce movement 106 in the artery wall 24.

In other embodiments, the catheter head either just collects radiation during operation or just emits radiation during operation, as in therapeutic applications. Further, the catheter head 58 is rotated 105 around its longitudinal axis in some examples.

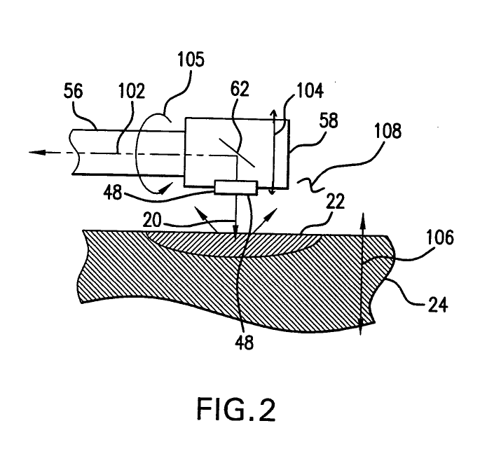

FIG. 3 shows the details of step 110 from FIG. 1 concerning the resection of the organ structure from the cadaver or other done, according to one embodiment. Specifically, in step 110A, a target artery is identified. In the example of a spectroscopic system for the identification of atherosclerotic lesions, a coronary artery from the donor is identified where possible lesions are present. Then, in step 110B, the artery is screened to identify a diseased section that exhibits one or multiple lesions. In step 110C, the artery including the diseased section is resected.

Specific examples of arterial conduits include the coronary arteries, carotid arteries, aorta, vertebral, basilar, cerebral, femoral, and iliac arteries. Examples of important venous conduits include saphenous, popliteal, femoral, jugular veins.

In other examples, other tubular organ structures are resected to be analyzed by the medical instrumentation. For example, a section of the esophagus, stomach, small, large intestine, anus, or rectum is resected from the cadaver. In still other examples, the nasopharynx, pharynx, larynx, bronchial tubes may be resected. Other portions of the genital or urinary may be resected including vagina, urethra, cervix, uterus, fallopian tube, ureters, renal pelvis. Alternative conduits such as Eustachian tube, biliary duct, pancreatic duct, salivary ducts, may also be resected.

As opposed to tubular organs or conduits, other solid organs or organ groups may be resected. These include, but are not limited to bone, cartilage, liver, spleen, kidney, skin, brain, spinal cord, nerve, muscle, pancreas, gall-bladder, heart, breast, prostate, thyroid, larynx, ovary, lung.

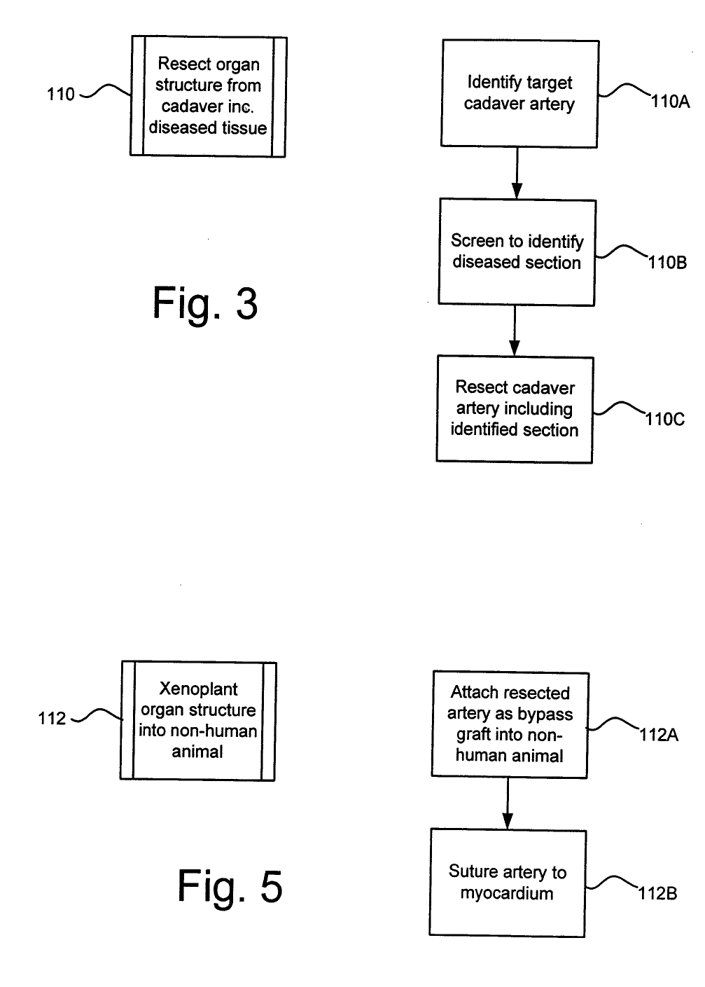

FIG. 4 is a flow diagram illustrating the detailed steps associated with the preparation of the resected artery of step 111 of FIG. 1, in one embodiment.

Specifically, in the present embodiment, pericardial and myocardial tissue is stripped away from the resected artery section in step 111A. The epicardial fat, however, is left on the artery in step 111B. It is believed that leaving the epicardial fat is important to maintain optical characteristics in the resected artery that are consistent with a living human patient.

In step 111C, the proximal and distal ends of the artery segment are freed from other tissue. This is a common step associated with bypass surgery in order to prepare the artery segment for anastomosis.

In step 111E, an assessment is made as to whether the proximal and/or distal ends of the resected artery segment are diseased. Typically, it is difficult to perform anastosmosis on diseased artery ends. If the ends are diseased, then in step 111F graft segments are added to the diseased ends in order to provide good grafting into the animal.

In step 111G, any bleeders on the artery are addressed. In one example, the artery can be encased in wax to stop the bleeders. Superglue could also be used.

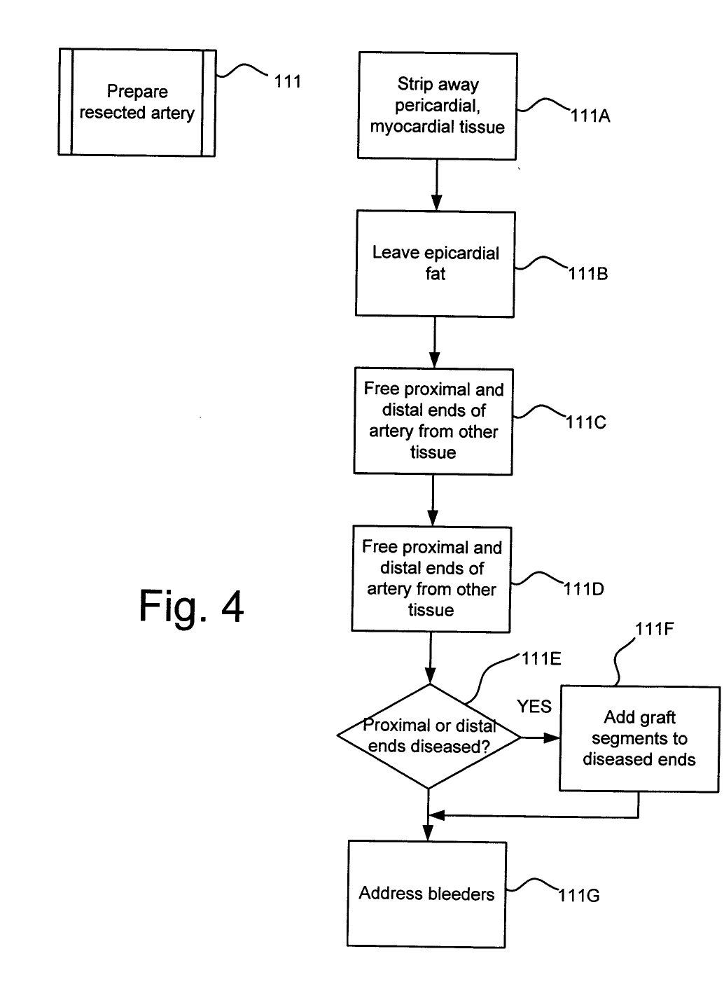

FIG. 5 illustrates the details associated with the xenoplantation of the organ structure into the non-human animal of step 112 in FIG. 1. Specifically, in step 112A, the resected artery is attached to the non-human animal. Presently, the graft is orthotropic, although heterotrophic grafts are used in other embodiments. Specifically, the graft is attached as a bypass graft to the heart of the model animal.

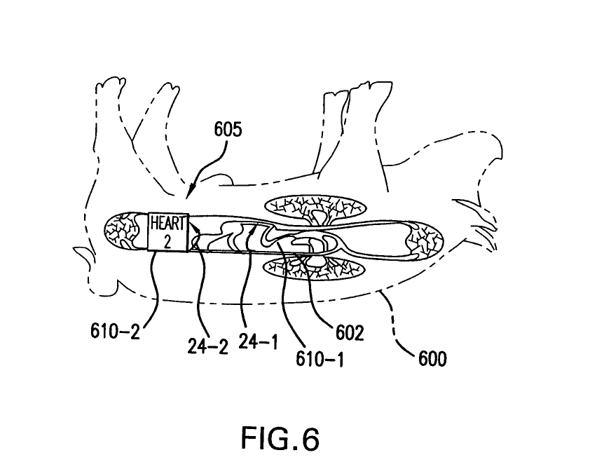

FIG. 6 shows this approach. Here, the resected human artery 24-1 is inserted into the circulatory system 602, and in one example attached to a heart 610-1, of the non human animal 600. The human artery 24-1 bridges a section of the animal's circulatory system in order to be exposed to the animal's blood flow.

The advantage of orthotopic grafting is that the human organ structures, such as the arteries, are subjected to an environment that is very similar to the environment in humans. In the example of grafting human coronary arties into a bypass graft on the non-human animal, the arteries subjected to a mechanical environment similar to that from which they came. This enables drug testing and/or instrumentation testing.

FIG. 6 also shows an alternative approach. Here, the resected human artery 24-2 is attached to a heart 610-2 that has been removed from another animal. This heart 610-2, with the human artery graft 24-2, is then heterotopically transplanted into the animal 600 of the animal model such as into the animal's abdomen 605. This procedure has advantages in that it avoids the need to operate on the animal's functioning heart 610-1. This makes the procedure easier for the surgeon to perform and typically improves the post operation viability of the animal 600.

Returning to FIG. 5, then, in step 112B, the artery is sutured to the myocardium of the pig heart. In some applications, how the artery is sutured is relevant. For example, in stent testing, it is sometimes important the stent is deployed at a certain curvature of artery. The appropriate curvature is implemented in these situations during this tack-down step of the xenotransplantation.

After the grafting of the artery to the non-human animal, additional steps can be performed to render the artery viable, if required for long-term study. For example, the vaso vasorum of the artery segment can be connected to receive blood flow. Also, the nerves can be attached if functional information is required.

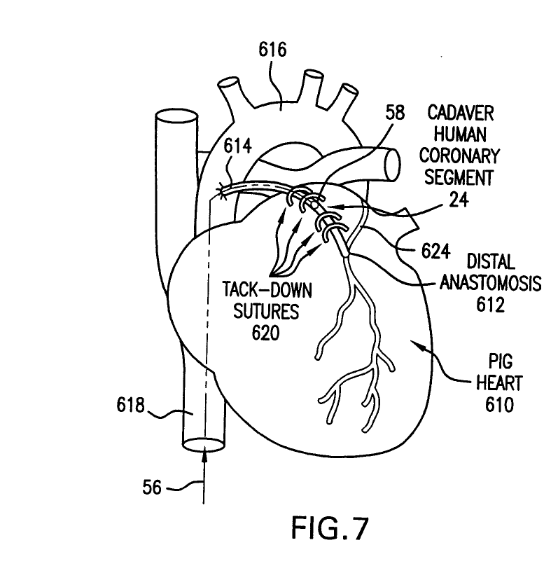

FIG. 7 shows an exemplary pig heart with the cadaver coronary segment 24. Specifically, the pig heart includes its native artery tree 624. A cadaver coronary artery segment 24, according to the invention, is attached. In the illustrated configuration, it is attached as a bypass graft. A proximal anastomosis 614 to the aorta at the base of the aortic arch 616 is made. The distal anastomosis 612 is made to the coronary artery 624 of the pig heart 610.

In other implementations, other attachment methods can be used. For example, an artery-to-artery path or a loop can also be used.

To ensure that the coronary artery segment 24 is exposed to the same sort of mechanical environment as a native coronary artery, tack down sutures 620 are used to attach the human coronary artery segment 622 to the pig heart 610.

Then, the probe, such as catheter, instrumentation, as illustrated in for example FIG. 2, is then used to either analyze or treat the human coronary segment 24. Specifically, the catheter 56, in one example, is advanced up the descending aorta 618 to the proximal anastomosis 614, thereafter, the catheter head is moved down the coronary artery segment 24.

When positioned, the catheter head 58 of the catheter 56 is used to analyze or treat the human coronary artery segment 24. This coronary artery segment 24 exists in a mechanical environment similar to a native human coronary artery due to the operation of the pig heart 610 and specifically, the mechanical motion from its pumping action and the blood flow environment the human coronary artery segment due to its anastomosis to the pig's circulatory system.

In other implementations, the treatment that is performed by the medical instrumentation is the placement of the stent in the human coronary segment. As a result, the efficiency with which the stent can be placed in the coronary artery segment can be tested to thereby assess the medical instrumentation.

With long term viability of the coronary artery segment, then the stent's long term performance can be further assessed during the subsequent histological examination after animal sacrifice.

Further, although the present invention is described in the context of the attachment of an organ structure to a circulatory system, attachment of organ structures to the genital urinary tracks, such as the kidneys, are performed in other implementations. In still other examples, thermography is performed on the human coronary artery segment 24. Further, treatment of abdominal aorta aneurisms can be tested by splicing an artery segment with an aneurism to the pig's circulatory system.

Moreover, the present invention is also applicable to the testing of drugs using instrumentation-based testing. For example, in one protocol, statin or similar drug is administered to the patient and then the efficacy of the drug is tested using the spectroscopic probe to analyze the intimae of blood vessels to find atherosclerotic lesions or plaque and assess their progress and response to the drug.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for using medical instrumentation including a probe, the method comprising:
inserting the probe into a non-human animal having a human tissue graft;
moving the probe to the human tissue graft; and
analyzing and/or treating the human tissue graft using the probe.

2. A method as claimed in claim 1, further comprising assessing a safety of the medical instrumentation.

3. A method as claimed in claim 1, further comprising assessing a performance of the medical instrumentation.

4. A method as claimed in claim 1, further comprising using the medical instrumentation in the training of operators of the medical instrumentation.

5. A method as claimed in claim 1, further comprising grafting the human tissue into the non-human animal, wherein the human tissue is a vessel segment.

6. A method as claimed in claim 1, further comprising grafting the human tissue into the non-human animal, wherein the human tissue is a blood vessel segment exhibiting an atherosclerotic lesion.

7. A method as claimed in claim 1, wherein the step of moving the probe to the human tissue graft comprises moving the probe through the body of the non-human animal.

8. A method as claimed in claim 1, wherein the step of moving the probe to the human tissue graft comprises moving the probe through blood vessels of the non-human animal.

9. A method as claimed in claim 1, wherein the step of analyzing and/or treating the human tissue graft using the probe comprises detecting a spectral response of the human tissue graft.

10. A method as claimed in claim 1, wherein the step of analyzing and/or treating the human tissue graft using the probe comprises detecting a spectral response of lesions in the human tissue graft.

11. A method as claimed in claim 1, wherein the step of analyzing and/or treating the human tissue graft using the probe comprises exposing the human tissue graft to optical energy from the probe.

12. A method as claimed in claim 1, wherein the human tissue graft is a tubular organ structure from a human.

13. A method as claimed in claim 1, wherein the probe includes a stent for placement in the human tissue graft.

14. A method as claimed in claim 1, further comprising analyzing the human tissue graft after the step of analyzing and/or treating the human tissue graft using the probe.

15. A method as claimed in claim 14, wherein the step of analyzing the human tissue graft includes sacrificing the non-human animal.

16. A method as claimed in claim 1, further comprising administering an enhancement agent to the non-human animal to affect a result of the analyzing and/or treating the human tissue graft using the probe.

17. An animal model for the use of medical instrumentation having a probe, the animal model comprising live non-human animals into which human tubular organ structures have been grafted.

18. An animal model as claimed in claim 17, wherein the animal model is used to assess a performance of the medical instrumentation.

19. A method as claimed in claim 17, further comprising using the medical instrumentation in the training of operators of the medical instrumentation.

20. An animal model as claimed in claim 17, wherein the human tubular organ structures include human blood vessels.

21. An animal model as claimed in claim 20, wherein the human blood vessels are grafted onto a heart of the non-human animal as a bypass graft.

22. An animal model as claimed in claim 17, wherein the human tubular organ structures include coronary arteries exhibiting atherosclerotic lesions.

23. An animal model as claimed in claim 17, wherein the human tubular organ structures are grafted onto or into an organ system of the non-human animal.

24. An animal model as claimed in claim 17, wherein the human tubular organ structures include human tissue surrounding the organ structures

25. An animal model as claimed in claim 17, wherein the human tubular organ structures are non-viable.

26. An animal model as claimed in claim 17, wherein the human tubular organ structures are viable.

27. An animal model as claimed in claim 17, wherein a blood supply to the human tubular organ structures from the non-human animals is provided to promote the viability of the human tubular organ structures.

28. An animal model as claimed in claim 17, wherein anticlotting agents are administrated to the non-human animals to promote a viability of the human tubular organ structures.

29. An animal model as claimed in claim 17, wherein immuno suppressants are administrated to the non-human animals to promote a viability of the human tubular organ structures.

30. An animal model as claimed in claim 17, wherein the human tubular organ structures are orthotopically xenoplanted into the non-human animal.

31. An animal model as claimed in claim 17, wherein the probe performs diagnostic functions.

32. An animal model as claimed in claim 17, wherein the probe performs therapeutic functions.

33. An animal model as claimed in claim 32, wherein the probe is used to place a stent.

34. An animal model comprising live non-human animals into which human vascular tissue has been grafted.

35. An animal model as claimed in claim 34, wherein the human vascular tissue includes human blood vessels.

36. An animal model as claimed in claim 35, wherein the human blood vessels are grafted onto a heart of the non-human animal as a bypass graft.

37. An animal model as claimed in claim 34, wherein the human vascular tissue is diseased.

38. An animal model as claimed in claim 34, wherein the human vascular tissue exhibits atherosclerotic lesions.

39. An animal model as claimed in claim 34, wherein tissue surrounding the human vascular tissue is grafted into the non human animal.

40. An animal model as claimed in claim 34, wherein the human vascular tissue is non-viable.

41. An animal model as claimed in claim 34, wherein the human vascular tissue is orthotopically xenoplanted into the non-human animal.

42. An animal model as claimed in claim 34, wherein the human vascular tissue is viable.

43. An animal model as claimed in claim 34, wherein a blood supply to the human vascular tissue from the non-human animals is provided to promote the viability of the human vascular tissue by vascular anastomosis of a blood supply for the vascular tissue.

44. An animal model as claimed in claim 34, wherein anticlotting agents are administrated to the non-human animals to promote a viability of the human vascular tissue.

45. An animal model as claimed in claim 34, wherein immuno suppressants are administrated to the non-human animals to promote a viability of the human vascular tissue.

46. An animal model comprising live non-human animals into which human cadaver tissue has been grafted.

47. An animal model as claimed in claim 39, wherein the human cadaver tissue includes human blood vessels.

48. An animal model as claimed in claim 39, wherein the human cadaver tissue includes coronary arteries exhibiting atherosclerotic lesions.

49. An animal model as claimed in claim 39, wherein the human cadaver tissue is grafted onto an organ system of the non-human animal.

50. An animal model comprising live non-human animals into which human organ structures have been orthotopically grafted.

51. An animal model as claimed in claim 50, wherein the human organ structures include human blood vessels.

52. An animal model as claimed in claim 51, wherein the human blood vessels are grafted onto a heart of the non-human animal as a bypass graft.

53. An animal model as claimed in claim 50, wherein the human organ structures include coronary arteries exhibiting atherosclerotic lesions.

54. An animal model as claimed in claim 50, wherein the human organ structures include human tissue surrounding the human organ structures.

55. An animal model as claimed in claim 50, wherein the human organ structures are non-viable.

56. An animal model as claimed in claim 50, wherein the human organ structures are viable.

57. An animal model as claimed in claim 50, wherein a blood supply to the human organ structures from the non-human animals is provided to promote the viability of the human organ structures.

58. An animal model as claimed in claim 50, wherein anticlotting agents are administrated to the non-human animals to promote a viability of the human organ structures.

59. An animal model as claimed in claim 50, wherein immuno suppressants are administrated to the non-human animals to promote a viability of the human organ structures.

60. An animal model as claimed in claim 50, wherein the human organ structure is analyzed after sacrificing the non-human animal.

61. An animal model comprising live non-human animals into which human organ structures have been heterotopically grafted to simulate motion associated with a cardiovascular system.

62. An animal model as claimed in claim 61, wherein the human organ structures include vascular tissue.

63. An animal model as claimed in claim 61, wherein the human organ structures include organ structures from human hearts.

64. An animal model as claimed in claim 61, wherein the human organ structures have been grafted to receive flowing blood from circulatory systems of the non-human animals.

65. A medical testing method comprising:
resecting a human tubular organ structure;
grafting the human tubular organ structure into a non human animal; and
using a medical probe on the human tubular organ structure in the non human animal.

66. A method as claimed in claim 65, wherein the step of resecting the human tubular organ structure comprises resecting a human blood vessel.

67. A method as claimed in claim 65, wherein the step of resecting the human tubular organ structure comprises resecting a human artery.

68. A method as claimed in claim 65, wherein the step of grafting the human tubular organ structure comprises grafting human blood vessels onto a heart of the non-human animal.

69. A method as claimed in claim 65, wherein the step of grafting the human tubular organ structure comprises grafting a human blood vessel onto a heart of the non-human animal as a bypass graft.

70. A method as claimed in claim 65, wherein the step of resecting the human tubular organ structure comprising resecting a human blood vessel exhibiting atherosclerotic lesions.

71. A method as claimed in claim 65, wherein the step of grafting the human tubular organ structure comprises grafting the human tubular organ structure onto or into an organ system of the non-human animal.

72. A method as claimed in claim 65, wherein the step of resecting the human tubular organ structure comprises resecting a target human tubular organ structure and tissue surrounding the organ structure and the step of grafting the human tubular organ structure includes grafting the tissue surrounding the organ structure with the human tubular organ structure into the non human animal.

73. A method as claimed in claim 65, wherein the step of resecting the human tubular organ structure comprises resecting a non-viable human tubular organ structure.

74. A method as claimed in claim 65, wherein the step of resecting the human tubular organ structure comprises resecting a viable human tubular organ structure.

75. A method as claimed in claim 65, wherein the step of grafting the human tubular organ structure includes providing a blood supply to the human tubular organ structure from the non-human animal to promote the viability of the human tubular organ structure.

76. A method as claimed in claim 65, further comprising administering anticlotting agents to the non-human animal to promote a viability of the human tubular organ structure.

77. A method as claimed in claim 65, further comprising administering immuno suppressants to the non-human animal to promote a viability of the human tubular organ structure.

78. A method as claimed in claim 65, wherein the step of grafting the human tubular organ structure comprises orthotopically xenoplanting into the non-human animal.

79. A method as claimed in claim 65, wherein the step of using the medical probe on the human tubular organ structure comprises inserting the probe into the non human animal to perform diagnostic functions relative to the tubular organ structure.

80. A method as claimed in claim 65, wherein the step of using the medical probe on the human tubular organ structure comprises inserting the probe into the non human animal to perform therapeutic functions relative to the tubular organ structure.

81. A method as claimed in claim 80, wherein the step of using the medical probe on the human tubular organ structure comprises placing a stent with the probe.

82. A method as claimed in claim 65, further comprising assessing a safety of the medical probe.

83. A method as claimed in claim 65, further comprising assessing a performance of the medical probe.

84. A method as claimed in claim 65, further comprising training operators of the medical probe.

85. A method as claimed in claim 65, further comprising analyzing the human tubular organ structure after the step of using the medical probe.

86. A method as claimed in claim 85, wherein the step of analyzing the human tubular organ structure includes sacrificing the non-human animal.

87. A method as claimed in claim 65, further comprising administering an enhancement agent to non human animal prior to using the medical probe on the human tubular organ structure in the non human animal.

88. A medical testing method comprising:
resecting human vascular tissue;
grafting the human vascular tissue into a non human animal; and
performing medical testing on the human vascular tissue in the non human animal.

89. A method as claimed in claim 88, wherein the step of performing medical testing includes administering chemical agents to the non-human animal.

90. A method as claimed in claim 88, wherein the step of resecting human vascular tissue includes resecting a human blood vessel.

91. A method as claimed in claim 90, wherein the step of grafting the human vascular tissue comprises grafting the human blood vessel onto a heart of the non-human animal as a bypass graft.

92. A method as claimed in claim 88, wherein the step of resecting human vascular tissue includes resecting diseased human vascular tissue.

93. A method as claimed in claim 92, wherein the human vascular tissue exhibits atherosclerotic lesions.

94. A method as claimed in claim 88, wherein the step of resecting the human vascular tissue comprises resecting a blood vessel and surrounding tissue and the step of grafting the human vascular tissue includes grafting the blood vessel and the tissue surrounding the blood vessel into the non human animal.

95. A method as claimed in claim 88, wherein the step of grafting the human vascular tissue comprises grafting non-viable human vascular tissue.

96. A method as claimed in claim 88, wherein the step of grafting the human vascular tissue comprises orthotopically xenoplanting the human vascular tissue into the non-human animal.

97. A method as claimed in claim 88, wherein the step of grafting the human vascular tissue comprises grafting viable human vascular tissue.

98. A method as claimed in claim 97, wherein the step of grafting the human vascular tissue comprises providing a blood supply to the human vascular tissue from the non-human animal to promote the viability of the human vascular tissue by vascular anastomosis of a blood supply for the vascular tissue.

99. A method as claimed in claim 88, further comprising administering anticlotting agents to the non-human animal to promote a viability of the human vascular tissue.

100. A method as claimed in claim 88, further comprising administering immuno suppressants to the non-human animal to promote a viability of the human vascular tissue.

101. A medical testing method comprising:
resecting human cadaver tissue;
grafting the human cadaver tissue into a non human animal; and
performing medical testing on the human cadaver tissue in the non human animal.

102. A method as claimed in claim 101, wherein the step of resecting the human cadaver tissue includes resecting human blood vessels.

103. A method as claimed in claim 101, wherein the step of resecting the human cadaver tissue includes resecting coronary arteries exhibiting atherosclerotic lesions.

104. A method as claimed in claim 101, wherein the step of grafting the human cadaver tissue into the non human animal comprises grafting the human cadaver tissue onto an organ system of the non-human animal.

105. A medical testing method comprising:
resecting a human organ structure;
orthotopically grafting the human organ structure into a non human animal; and
performing medical testing on the human organ structure in the non human animal.

106. A method as claimed in claim 105, wherein the step of resecting the human organ structure comprises resecting human blood vessels.

107. A method as claimed in claim 106, wherein the step of orthotopically grafting the human organ structure into the non human animal grafting human coronary arteries onto a heart of the non-human animal.

108. A method as claimed in claim 105, wherein the step of resecting the human organ structure includes resecting a human coronary artery exhibiting atherosclerotic lesions.

109. A method as claimed in claim 105, wherein the step of resecting the human organ structure comprises resecting a blood vessel and surrounding tissue and the step of grafting includes grafting the blood vessel and the tissue surrounding the blood vessel into the non human animal.

110. A method as claimed in claim 105, wherein the step of resecting the human organ structure comprises resecting a non-viable human organ structure.

111. A method as claimed in claim 105, wherein the step of resecting the human organ structure comprises resecting a viable human organ structure.

112. A method as claimed in claim 105, wherein the step of orthotopically grafting the human organ structure into the non human animal comprises providing a blood supply to the human organ structure from the non-human animal to promote the viability of the human organ structure.

113. A method as claimed in claim 105, further comprising administering anticlotting agents to the non-human animal to promote a viability of the human organ structure.

114. A method as claimed in claim 105, further comprising administering immuno suppressants to the non-human animal to promote a viability of the human organ structure.

115. A medical testing method comprising:
resecting a human organ structure;
heterotopically grafting the human organ structure into a non human animal to simulate motion associated with a cardiovascular system; and
performing medical testing on the human organ structure in the non human animal.

116. A method as claimed in claim 115, wherein the step of resecting the human organ structure comprises resecting vascular tissue.

117. A method as claimed in claim 115, wherein the step of resecting the human organ structure comprises resecting a human heart.

118. A method as claimed in claim 115, wherein the step of grafting the human organ structure comprising attaching the human organ structure to receive flowing blood from a circulatory system of the non-human animal.
